(19) United States
(12) Patent Application Publication
(10) Pub. No.: US 2014/0120068 A1
(43) Pub. Date: May 1, 2014

(54) SOLUTIONS, SYSTEMS AND METHODS FOR CELL, TISSUE AND ORGAN PRESERVATION

(71) Applicant: Animal Cell Therapies, Inc., San Diego, CA (US)

(72) Inventors: Kathy Petrucci, La Jolla, CA (US); Chih-Min Lin, San Diego, CA (US)

(73) Assignee: Animal Cell Therapies, Inc., San Diego, CA (US)

(21) Appl. No.: 14/070,277

(22) Filed: Nov. 1, 2013

Related U.S. Application Data
(60) Provisional application No. 61/776,179, filed on Mar. 11, 2013.
(60) Provisional application No. 61/721,328, filed on Nov. 1, 2012.

(51) Int. Cl. A01N 1/02; A61F 2/02; A61F 2/24

(52) U.S. Cl. CPC A01N 1/0226; A61F 2/24; A61F 2/02
USPC 424/93.7; 435/1.1; 435/374; 435/1.3; 435/284.1; 623/3.29; 623/23.72

(57) ABSTRACT

This disclosure provides solutions, systems, and methods for cell, tissue, and/or organ preservation. Some preservation solutions may include any combination of a balanced salt solution, electrolytes, antibiotic agents, antimycotic agents, protease inhibitors, anti-oxidants, simple sugars, starches impermeant ions, uric acid and/or amino acids. Some preservation solutions may also include hydrolyzed collagen. The preservation solutions including hydrolyzed collagen may be used alone or as part of a kit to preserve cells, tissue, or organs. The solution may also be used in connection with one or more medical procedures, for example organ transplantation.

### RELATED APPLICATIONS

This application claims priority to U.S. Provisional Patent Application No. 61/721,328, filed Nov. 1, 2012, and to U.S. Provisional Patent Application No. 61/776,179, filed Mar. 11, 2013, both entitled Solutions, Systems and Methods for Cell, Tissue and Organ Preservation, the disclosures of which are incorporated by reference.

### BACKGROUND

The preservation of tissue and cells has many applications in biological sciences and medicine where such tissues and cells may be used for medical procedures, research and therapy such as organ, tissue or cell transplantation. However, because such cells and tissues are obtained from an organism, such as a human or animal, at some time prior to use of the cells or tissues, the cells or tissues must be preserved for some period of time while maintaining as close to normal structure and function as possible.

Preservation products and methods may be necessary, because tissue and cells that are removed from an organism quickly degrade. Lack of perfusion to the tissue, necessary for processes like metabolism and osmosis, can result in the degradation of the excised tissue over time and also make the tissue more susceptible to bacterial and fungal infection. After a period of time outside of the body, tissue and cells degrade to a point where they may be rendered unusable for their intended purposes and/or are no longer viable. Preservation products and methods that extend the viability of tissue and cells are desirable to allow additional time between collection and use.

One common method of preserving cells a tissue is to cool the cells and tissue to hypothermic temperatures, typically between 0° and 4° C., to slow the rate of metabolism. Cooling alone, however, may not an effective means of tissue and cell preservation in many cases. While cooling may slow the rate of metabolism, it does not stop it completely. Furthermore, cooling can inhibit the operation of cell membrane pumps that counteract the passive exchange of water and ions thereby making the cell susceptible to osmotic injury. Consequently, tissue preservation solutions are often used in conjunction with cooling to prolong tissue and cell viability. These solutions vary in composition but may include ions, buffer solutions, impermeants, and protease inhibitors. Tissue preservation solutions are generally designed to maintain cell morphology by minimizing cell and tissue swelling, maintaining ionic balance, preventing the development of acidosis, and removing and/or inhibiting the formation of free radicals. Where the organs are being preserved for transplantation, preservation solutions may also provide metabolic substrates and stimulate recovery upon reperfusion.

Where the cells and/or tissue are to be stored for a longer period of time, the cells (such as stem cells) and/or tissue may be frozen. The components of a preservation solution may vary in accordance with their intended usage. For example, a preservation solution formulated as a freezing media may contain components like antifreeze proteins or synthetic ice blockers that are not usually found in preservation solutions formulated as a transport media.

### BRIEF DESCRIPTION OF THE DRAWINGS

The above mentioned and other advantages of the present invention, and the manner of attaining them, will become more apparent and the invention itself will be better understood by reference to the following description of the embodiments of the invention taken in conjunction with the accompanying drawing, wherein:

FIG. 1 is a graph of cell count over time for cells stored in solutions with and without hydrolyzed collagen; and

FIG. 2 is a graph of cell viability over time for cells stored in solutions with and without hydrolyzed collagen.

FIG. 3A is a graph of cell viability over time for cells stored in solutions with and without hydrolyzed collagen.

FIG. 3B is a graph of cell viability for cells stored for one day in solutions with and without hydrolyzed collagen.

FIG. 3C is a graph of cell viability for cells stored for six to seven days in solutions with and without hydrolyzed collagen.

FIG. 3D is a graph of cell viability for cells stored for thirteen days in solutions with and without hydrolyzed collagen.

FIG. 3E is a graph of cell viability for cells stored for twenty to twenty-one days in solutions with and without hydrolyzed collagen.

FIG. 4 is a graph showing the percentage of cells attached in the samples of Example 6 and Table 6.

FIG. 5A is a graph showing atrium contractility of the hearts of Example 7 and Table 7.

FIG. 5B is a graph showing ventricle contractility of the hearts of Example 7 and Table 8.

FIG. 5C is a graph showing atria beats per minute of the hearts of Example 7 and Table 9.

FIG. 5D is a graph showing ventricle beats per minute of the hearts of Example 7 and Table 9.

FIG. 5E is a graph showing perfusion of the hearts of Example 7 and Table 9.

FIG. 5F is a graph showing perfusion color of the hearts of Example 7 and Table 9.

### SUMMARY

Embodiments of the invention include solutions, kits, and methods for preserving cells such as stem cells and other cells, tissues, and organs which prolong the viability of the cells, tissues, and organs. Preservative solutions which may be used in various embodiments may include a balanced isotonic solution and hydrolyzed collagen. In some embodiments, the preservative solution includes only these two components or essentially only these two components. In other embodiments, the preservative solution may optionally include other components, such as one or more of antibiotics, antimycotics, protease inhibitors, impermeant anions, antifreeze protein, and/or synthetic ice blocker. The balanced isotonic solution may include sodium, potassium, calcium, and chloride. The concentration of the hydrolyzed collagen may be between about 1 and about 10 mg/ml in some embodiments, though other concentrations may also be used.

The preservative solution may be provided as an aqueous solution or may be provided in a powder form to which water may be added to form the aqueous solution. In some embodiments, the preservative solution may be provided in a kit which may also include a container for storing an organ, tissue, or cells. The solution in the kit may be provided in aqueous or powder form. In some embodiments, the components of the preservative solution may be provided separately in the kit.

Methods of preserving organs, tissue and cells according to various embodiments may include harvesting an organ, tissue or cells and submerging the harvested organ, tissue or cells in a bath of the preservative solution. The method may further include cooling the organ, tissue or cells, such as to a temperature of about 0-37° C., for example.

Still other embodiments include methods of transplanting an organ, tissue or cells. The organ, tissue, or cells may be present in the preservative solution, and the method may include removing the organ, tissue, or cells from the preservative solution and providing the organ, tissue, or cells to the body of a recipient, such as by injecting or implanting the organ, tissue, or cells into the recipient.

### DETAILED DESCRIPTION

The following detailed description is exemplary in nature and is not intended to limit the scope, applicability, or configuration of the invention in any way. Rather, the following description provides practical illustrations for implementing exemplary embodiments of the present invention. Thus, for the purpose of promoting an understanding of the principles of the invention, reference will now be made to the embodiments illustrated in the examples and specific language will be used to describe the same. It will, nevertheless, be understood that no limitation of the scope of the invention is thereby intended; any alterations and further modifications of the described or illustrated embodiments, and any further applications of the principles of the invention as illustrated therein, are contemplated as would normally occur to one skilled in the art to which the invention relates.

This application discloses preservation solutions, preservation methods, and kits or systems for preservation of tissues, cells and/or organs, that prolong the viability of tissue, cells and organs after removal from the organism in which they originated. Embodiments include hydrolyzed collagen in solutions in which tissues, cells, and/or organs may be stored to preserve them for later use. It has been found that preservation solutions containing hydrolyzed collagen are unexpectedly effective in the preservation of such tissues, cells and/or organs, prolonging their viability and maintaining their morphology.

Hydrolyzed collagen has previously been used as a dietary supplement to promote joint, bone and skin health, as an ingredient in cosmetics, as well as an anti-freezing agent in ice cream manufacturing. In some contexts, hydrolyzed collagen may be referred to as collagen hydrosylate, collagen peptide, gelatin hydrosylate, hydrolyzed gelatin, or gelatin hydrolysate enzymatic. Hydrolyzed collagen can be produced from collagen found in bones, skin and connective tissue of animals according to known methods. Hydrolysis is performed on the collagen to break it into peptides having an average molecular weight of 1,000 to 10,000 Daltons, with the resulting composition being hydrolyzed collagen. Methods of obtaining hydrolyzed collagen are known in the art, as described, for example, in “The development of novel recombinant human gelatins as replacements for animal-derived gelatin in pharmaceutical applications,” Olsen D, et al., in Pasupuleti V K, Demain A (eds) Protein hydrolysates in biotechnology, Springer, The Netherlands (2010), as well as in Gelatin hydrolysate from blacktip shark skin prepared using papaya latex enzyme: Antioxidant activity and its potential in model systems, Phanat P Kittiphattanabawon et al., Food Chem 135(3):1118-26 (2012), PMID 22953833. A method of obtaining hydrolyzed collagen is also provided in U.S. Patent Publication Number 2008/0275140, for example. Hydrolyzed collagen may also be obtained commercially from companies such as Sigma-Aldrich, for example. In some embodiments, the hydrolyzed collagen may be sterile while in other embodiments sterility is not required.

The preservative solutions may be aqueous solutions and may include only saline and hydrolyzed collagen, for example. In some embodiments, the solution may also include additional electrolytes and/or a buffered solution. For example, in some embodiments the solutions may include a balanced salt solution (BSS), serving as a pH buffer, and hydrolyzed collagen. Examples of electrolytes which may be included in the solutions include one or more of sodium, potassium, chloride, and bicarbonate, for example. Examples of balanced salt solutions which may be used include saline, PBS (phosphate buffered saline), HBSS (Hank's Balanced Salt Solution) or other buffers. The preservative solutions may also include other optional components such as one or more of the following optional components: antibiotic agents, antimycotic agents, protease inhibitors, anti-oxidants, simple sugars, starches, impermeant ions, uric acid and/or amino acids. Examples of antibiotics which may be used include in the preservative solution include streptomycin, penicillin, ampicillin amphotericin B, gentamycin, and/or neomycin, for example. Examples of antimycotic agents which may be used in the preservative solutions include anti-PPLO agent tylosin and nystatin, for example. Examples of protease inhibitors which may be used in the preservative solutions include various protease inhibitor cocktails such as serine protease inhibitors, cysteine protease inhibitors, and metalloprotease inhibitors, for example. Examples of anti-oxidants which may be used in the preservative solutions include ascorbic acid, vitamin E, GSH, catalease, and superoxide dismutase, for example. Examples of simple sugars which may be used in preservative solutions include glucose and sucrose, for example. Examples of starches which may be used in the preservative solutions include hydroxyethyl starch, and dextrinized starch, for example. Examples of impermeant ions which may be used include lactobionate, sucrose, mannitol, glucose, gluconate, and dextran, for example. Examples of amino acids which may be used include essential amino acids and non-essential amino acids.

In some embodiments, the hydrolyzed collagen may be added to or provided in combination with a known preservative solution or media or storage solution. Examples of preservative solutions or media or storage solutions to which the hydrolyzed collagen may be added include, but are not limited to, HIBERNATE (available from Life Technologies), University of Wisconsin Cold Storage Solution (also known as ColStorSol or ViaSpan) and HTK (histidine-tryptophan-ketoglutarate).

In some embodiments, hydrolyzed collagen, also sometimes known as gelatin hydrolysate or gelatin, may be present in the preservative solution at various concentrations. For example, in some embodiments, the hydrolyzed collagen may be between about 0.1 mg/ml and about 100 mg/ml, or between about 0.5 and about 50 mg/ml, or between about 1 and about 25 mg/ml, or between about 1 and about 10 mg/ml. The use of preservative solutions including hydrolyzed collagen as described herein can maintain some or a portion of the cells (as individual cells or within the tissue or organ) in a viable condition for a period of time of up to 1 day, 2 days, 3 days, 4 days, 5 days, 6 days, 1 week, 2 weeks, 3 weeks, or 4 weeks, for example. In some embodiments, some or all of the cells may remain viable for an even longer period of time. At the end of this time period, enough cells may remain viable for the cells, tissues, or organs to remain effective for their intended purpose.

It should be understood that a viable cell is a cell that is alive and functional. Viability may be determined using known techniques such as the Trypan Blue exclusion test of cell viability. The preservative solutions described herein preserve the cells, tissues, and organs in a viable state, and may be considered to be stabilizing solutions. This is to be distinguished from cell fixation solutions and processes in which the cells are preserved to prevent decay but are dead and are no longer viable.

In some embodiments, the solution may be used for the storage and/or transportation of cells, tissues, and/or organs after removal from the organism. In such cases, the preservative solution may be at a temperature above freezing, such as a temperature of between about 30° F. to about 50° F. prior to placement of the cells, tissues, and/or organs in the solution, and/or the stored cells, tissues, and/or organs may be stored at this temperature after placement in the preservative solution. In some embodiments, the container holding the cells, tissues, or organs in the solution may be placed on ice or into an ice bath, such as a container holding an organ which will be used for transplantation into another individual. The use of a reduced temperature may improve the effectiveness of the solution at preservation. The cells, tissues, and/or organs may be bathed or submerged in the solution, such as in a storage and/or transportation container. In some embodiments, the cells may be stirred or injected into the storage solution.

In some embodiments, the solution may also be used for storage of tissue or cells in a frozen state, such as at a temperature of between about −80° C. to about −196° C., for example, though other freezing temperatures may also be used. In such embodiments, the solutions may also include antifreeze proteins and/or synthetic ice blockers. Examples of antifreeze proteins and cryprotectants that may be used include glycoproteins, DMSO, and serum, for example. Examples of ice blockers that may be used include 1,3-cyclohexanediol and 1,4-cyclohexanediol, for example. In embodiments in which the preservative solution is used for freezing the cells, tissues, and/or organs, the same solution that is used for frozen storage of the cells for tissues may also be used for storage before and/or after freezing. For example, at the time of collection of the cells, tissues, and/or organs, the cells, tissue and/or organs may be deposited into and transported in the preservative solution prior to freezing, and then may be subsequently frozen, all in the same solution. Likewise, after thawing of the tissue and/or cells in the solution, they may be stored in the solution at a temperature above freezing, such as between about 30° F. and about 50° F., for an additional period of time, such as at least one day, at least two days, or at least five days, at least one week, at least two weeks, or longer, without changing the solution.

When the storage solution is used for preserving cells, such as stem cells or other cells, tissues, and/or organs, the cells, tissues, and/or organs may be submerged in the solution and/or flushed with the solution. For example, after removal from the donor organism (such as an animal or human) in which the cells, such as stem cells or other cells, tissues, or organs were growing naturally, they may be flushed with the solution and/or directly placed into a container containing the preservation media. If the cells, tissues, and/or organs are to be stored without freezing, they may be stored and/or transported in the solution to a location at which they will be further processed and/or used. In some embodiments, the cells such as stem cells or other cells, tissues, and/or organs may be removed from the solution and washed, flushed and/or perfused with a different solution, such as saline or a buffered solution, prior to use, such as to remove all or substantially all of the preservative solution. For example, when an organ is to be used for transplantation, it may be removed from the preservative solution and flushed and/or perfused prior. The organ may further be warmed to an appropriate temperature. The organ may then be implanted into a recipient.

Various embodiments also include kits for storing and preserving cells such as stem cells or other cells, tissues, and/or organs. The kits may include a container in which the cells, tissues, and/or organs may be stored, and a preservative solution as described herein. Examples of appropriate containers include cell cryovials and tissue/organ containers. The solution may be provided in the storage container as part of the kit, or may be provided in a separate container and may be added to the storage container by the user prior to depositing the cells, tissues, and/or organs in the container. The storage container and solution may be individually or separately sealed in a package to maintain sterility prior to use or otherwise provided in a sterile manner.

In some embodiments, the preservative solution may be made by an individual, such as by a user of the preservative solution, by adding hydrolyzed collagen to a solution, such as a commercially available solution, such as a balanced isotonic solution or a cell preservation solution, and optionally further adding other components as described herein.

In some embodiments, the preservative solution may be provided in a dry form, such as a sterilized powder form, which may be created by lyophilization or free-drying, for example. In such embodiments, the user may add the powder or other dry component including hydrolyzed collagen to water, saline, or a balanced isotonic solution, or a preservative solution, for example, to form the aqueous preservative solution.

In some embodiments, the preservative solution is provided to a user with some of the components packaged separately from others. For example, the basal media, which may be saline or a balanced isotonic solution, may be provided in a first container. The hydrolyzed collagen may be provided separately in a second container. In embodiments in which the preservative solution also includes an antibiotic and/or antimycotic, the antibiotic and/or antimycotic may be provided in the second container along with the hydrolyzed collagen, or it may be provided separately in a third container. In this way, the preservation solution may be more easily stored prior to use, by storing the hydrolyzed collagen and the antibiotic and/or antimycotic in a refrigerated environment, for example, while the basal media may not require refrigeration.

A variety of explanted cells, tissues, and organs may be preserved and/or frozen using the preservative solutions described herein. Examples include, but are not limited to, isolated cells, such as stem cells including mesenchymal cells, bone marrow, islet cells, tissues such as umbilical cords, cartilage, corneas, skin, heart valves, ligaments, veins, and arteries, and organs such as hearts, lungs, livers, kidneys, pancreas, and intestine, for example.

Specific examples have been provided describing various uses of a preservation solution including hydrolyzed collagen. These examples are provided for the purpose of illustration only and are not meant to limit the scope of the invention. One skilled in the art will appreciate that the preservation solution including hydrolyzed collagen can be used in many different settings and applications.

Specific examples have been provided describing various uses of a preservation solution including hydrolyzed collagen. These examples are provided for the purpose of illustration only and are not meant to limit the scope of the invention. One skilled in the art will appreciate that the preservation solution including hydrolyzed collagen can be used in many different settings and applications.

### EXPERIMENTAL

The following experiments were conducted with liver cells, umbilical cords, mesenchymal stem cells and chicken embryo hearts. In each of these examples, multiple samples of tissues and/or cells were preserved with various preservation solutions, with or without hydrolyzed collagen, and stored at 4° C. The hydrolyzed collagen used in these examples was gelatin hydrolysate enzymatic obtained from Sigma-Aldrich (Cat No. G0262). The antibiotic/antimycotic was a 10× antibiotic/antimycotic obtained from Invitrogen (Cat No. 15240-062). The protease inhibitor was a protease inhibitor cocktail obtained from Sigma Aldrich (Cat No. S8830). In examples 1, 2, and 4, after storage in the solutions at 4° C., the organs were enzymatic digested for cell isolation and a cell count was then determined for each sample using the Trypan Blue Exclusion method, and the effectiveness of each solution was compared. Unless otherwise indicated, the cell count results shown in each example are the average of the results for the multiple samples measured for each preservation solution and each time period.

### Example 1

Liver samples were collected from chickens on embryonic day eight. The samples were weighed and then stored for seven days at 4° C. in various preservation solutions as shown in Table 1. With regard to preservation solutions of samples 1 and 2, the Hanks balanced salt solution (HBSS) and the antibiotic/antimycotic were present at the same concentration (as provided by the manufacturer) in each sample. Similarly, with regard to preservation solutions of sample 3 and 4, the HBSS and the protease inhibitor were present at the same concentration in each sample. At the end of seven days, the organs were enzymatic digested for cell isolation and a cell count was obtained from each sample, and this count was then divided by the weight of the corresponding sample to yield a cells/g metric. The results are shown in Table 1:

TABLE 1
Cell count in preservation solutions with and without gelatin

| Sample | Solution | Cell count (cells/g) |
|---|---|---|
| 1 | HBSS, antibiotic/antimycotic | 16.12 × 10⁶ |
| 2 | HBSS, antibiotic/antimycotic, | 26.3 × 10⁶ |
| | hydrolyzed collagen (1 mg/ml) | |
| 3 | HBSS, protease inhibitor | 14.88 × 10⁶ |
| 4 | HBSS, gelatin, protease inhibitor | 33.81 × 10⁶ |

It was found that the preservation solution with hydrolyzed collagen yielded a much higher cells/g count than the identical solution without hydrolyzed collagen. At the end of the 7 day time period, the cells were examined microscopically and it was observed that the cells of samples preserved in solutions with hydrolyzed collagen exhibited cell morphology were more uniform and exhibited less swelling than cells in solutions without hydrolyzed collagen.

### Example 2

Canine umbilical cords were collected at the time of Cesarean section and were preserved in the varying preservation solutions of Table 2, as shown below, and stored at 4° C. for periods of time ranging between six and seven days. The length of each cord sample was then measured and the mesenchymal cells were harvested from each sample. Vessels and capillaries were removed from the cord and each cord was incubated in an enzymatic solution to dissociate the tissue to allow for collection of the mesenchymal stem cells by centrifugation. Further description of the process of harvesting the mesenchymal cells may be found in Methods in cell biology, 2008, Vol. 86, at pages 101-119. A cell count of the mesenchymal stem cells was collected and tissue viability was calculated for each sample by dividing the cell count by the length of the cord sample. The results were collected and are shown in Table 2 below:

TABLE 2
Viability of cells in various preservation solutions
with and without gelatin

| | | Viability |
|---|---|---|
| Sample | Solution | (cells/cm) |
| 3 | HBSS, antibiotic/antimycotic | 4.7 × 10⁵ |
| 4 | HBSS, antibiotic/antimycotic | 3.3 × 10⁵ |
| 5 | HBSS, antibiotic/antimycotic | 3.1 × 10⁵ |
| 6 | HBSS, antibiotic, antimycotic | 7.09 × 10⁵ |
| 7 | HBSS, antibiotic/antimycotic, protease inhibitor | 1.08 × 10⁵ |
| 8 | HBSS, antibiotic/antimycotic, hydrolyzed | 7.5 × 10⁵ |
| | collagen (5 mg/ml) | |
| 9 | HBSS, antibiotic/antimycotic, protease inhibitor, | 1.3 × 10⁶ |
| | hydrolyzed collagen (1 mg/ml) | |
| 10 | HBSS, antibiotic/antimycotic, protease inhibitor, | 1.45 × 10⁶ |
| | hydrolyzed collagen (4 mg/ml) | |
| 11 | HBSS, antibiotic/antimycotic, protease inhibitor, | 1.04 × 10⁶ |
| | hydrolyzed collagen (5 mg/ml) | |

It was found that preservation solutions with hydrolyzed collagen yielded a substantially higher cells/cm count compared to identical solutions without hydrolyzed collagen. Indeed, even after storage for 6 or 7 days in the preservative solutions including hydrolyzed collagen, the cells/cm was comparable to the number of cells/cm typically obtained from freshly harvested umbilical cords of the same type, which the inventors have previously found to be between about 1.0 and 1.5×10⁶ cells/cm. The isolated mesenchymal cells were then examined microscopically and it was observed that cell morphology of all solutions containing hydrolyzed collagen was more uniform, having a regular spindle-shape, as compared to varied results among solutions without hydrolyzed collagen.

### Example 3

At the end of the experiment in Example 2, the mesenchymal stem cells were collected from each of samples 3-11, placed in a culture, and then harvested when the culture reached 70-90% confluency. The cells that were preserved in solutions with hydrolyzed collagen reached confluency three or more days earlier than cells preserved in solutions without hydrolyzed collagen.

### Example 4

Mesenchymal stem cells obtained from canine umbilical cords as described above with regard to example 2 were preserved in solutions with and without hydrolyzed collagen as shown in Table 3 below. Six samples of 1×106 mesenchymal stem cells were suspended in two preservation solutions consisting of the same concentration of HBBS (identified as PBS in Table 3). The samples identified as PBS in Table 3 included only HBBS in the solution, while the sampled identified as Gelatin also included hydrolyzed collagen at 1 mg/ml. Each sample was stored at 4° C., and a cell count was collected every 24 hours. The results are shown below in Table 3.

TABLE 3
Viability of cells in preservation solutions with and without gelatin between 24 hours and 120 hours

| | | 24 hrs | | 48 hrs | | 72 hrs | | 96 hrs | | 120 hrs | |
|---|---|---|---|---|---|---|---|---|---|---|---|
| | | Cell Count | Viability | Cell Count | Viability | Cell Count | Viability | Cell Count | Viability | Cell Count | Viability |
| | | (×105) | (%) | (×105) | (%) | (×105) | (%) | (×105) | (%) | (×105) | (%) |
| PBS | 1 | 5.35 | 78 | 4.8 | 82 | 3.7 | 63 | 2.3 | 41 | 1.15 | 21 |
| | 2 | 6.45 | 81 | 6.05 | 71 | 4.2 | 71 | 3.15 | 48 | 1.2 | 22 |
| | 3 | 6.25 | 87 | 6.7 | 82 | 3.2 | 60 | 2.4 | 41 | 1.1 | 17 |
| Gelatin | 1 | 7.3 | 84 | 8.05 | 78 | 8.8 | 84 | 8 | 63 | 5.4 | 51 |
| (1 mg/ml) | 2 | 8.6 | 86 | 8.1 | 84 | 6.75 | 81 | 8.3 | 72 | 3.65 | 50 |
| | 3 | 8.05 | 85 | 7.8 | 81 | 8.2 | 85 | 7.7 | 71 | 6.8 | 50 |

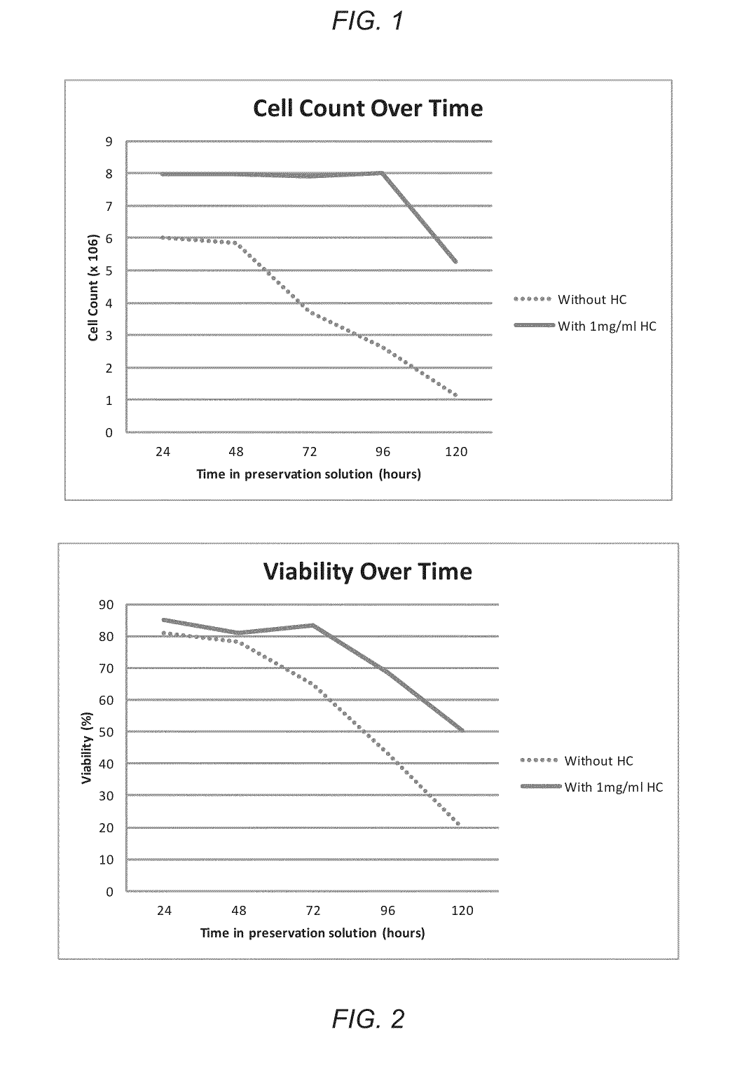

The cell count for each time period was averaged for the three samples with and without hydrolyzed collagen and the average cells counts were plotted over time in FIG. 1. It can be seen that the average cell count over time of the samples preserved in solutions with hydrolyzed collagen was higher than the average cell count of solutions without hydrolyzed collagen.

The percent viability was likewise averaged at each time period for the three samples with, and the three samples without, hydrolyzed collagen and the average viability was plotted over time in FIG. 2. It can be seen that the average cell viability over time of the samples preserved in solutions with hydrolyzed collagen was higher than that of samples preserved in solutions without hydrolyzed collagen.

Cells from each sample were analyzed microscopically at the end of the experiment. It was found that the cells preserved in solutions with hydrolyzed collagen exhibited no swelling, while cells preserved in solutions without hydrolyzed collagen exhibited swelling.

### Example 5

Canine umbilical cords were preserved in varying solutions using a method similar to the method of Example 2. Canine umbilical cords were collected at the time of Cesarean section and were placed into the preservation solutions of Table 5, as shown below, and stored at 4° C. for periods of time ranging between one and twenty-one days. The length of each cord sample was then measured and the mesenchymal cells were harvested from each sample. Vessels and capillaries were removed from the cord and each cord was incubated in an enzymatic solution to dissociate the tissue to allow for collection of the mesenchymal stem cells by centrifugation. Further description of the process of harvesting the mesenchymal cells may be found in Methods in cell biology, 2008, Vol. 86, at pages 101-119. A cell count of the mesenchymal stem cells was collected and tissue viability was calculated for each sample by dividing the cell count by the length of the cord sample. The results were collected and are shown in Table 5 below:

TABLE 5
Viability of cells in preservation solutions with and without
gelatin between 1 and 21 days

| | Time in | | Viability |
|---|---|---|---|
| Sample | Solution (days) | Solution | (cells/cm) |
| 1 | 1 | HBSS | 1.15 × 10⁶ |
| 2 | 1 | Hibernate | 1.05 × 10⁶ |
| 3 | 1 | HBSS, gelatin | 1.19 × 10⁶ |
| 4 | 6-7 | HBSS | 2.9 × 10⁵ |
| 5 | 6-7 | Hibernate | 6.6 × 10⁵ |
| 6 | 6 | HBSS, gelatin | 1.56 × 10⁶ |
| 7 | 6 | Hibernate, gelatin | 2.05 × 10⁶ |
| 8 | 13 | HBSS | 1.42 × 10⁵ |
| 9 | 13 | Hibernate | 1.35 × 10⁵ |
| 10 | 13 | HBSS, gelatin | 5.32 × 10⁵ |
| 11 | 13 | Hibernate, gelatin | 5.95 × 10⁵ |
| 12 | 20 | HBSS | 4.9 × 10⁴ |
| 13 | 21 | HBSS, gelatin | 2.58 × 10⁵ |

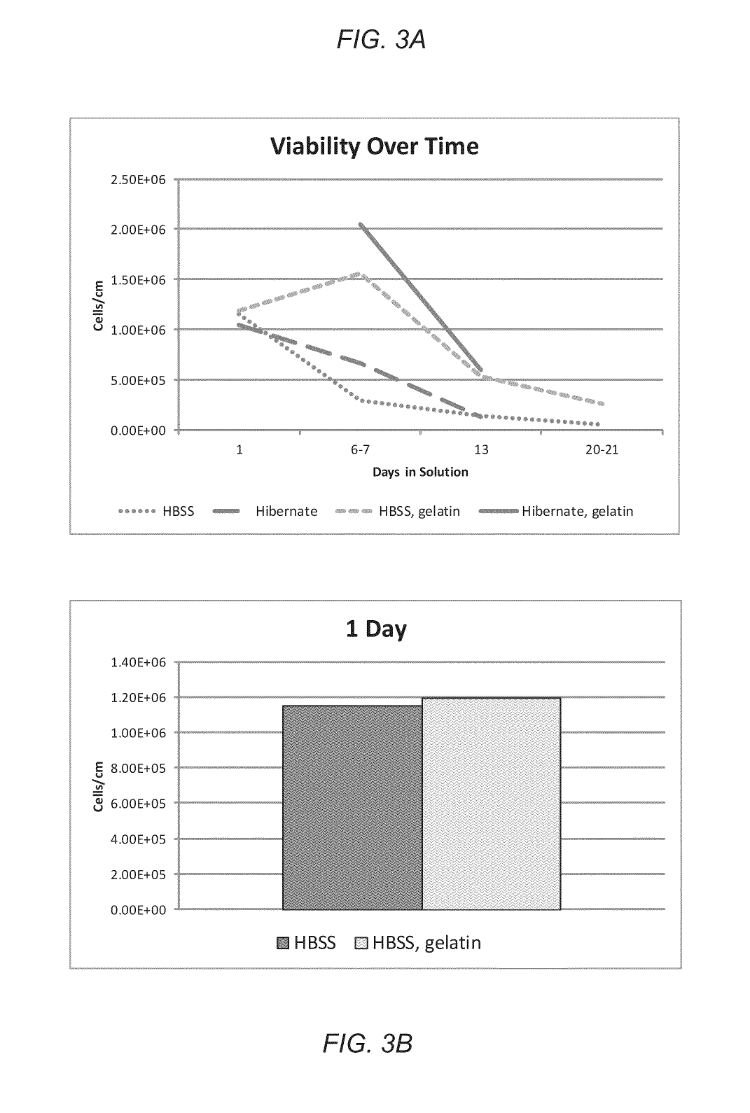

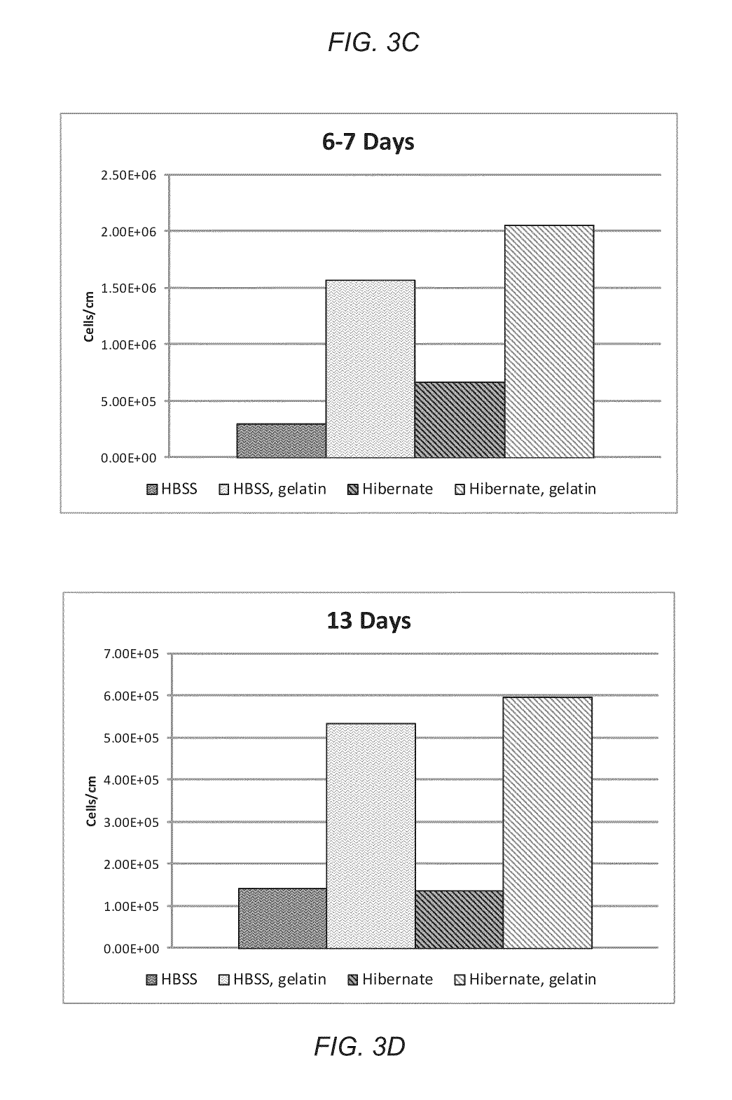

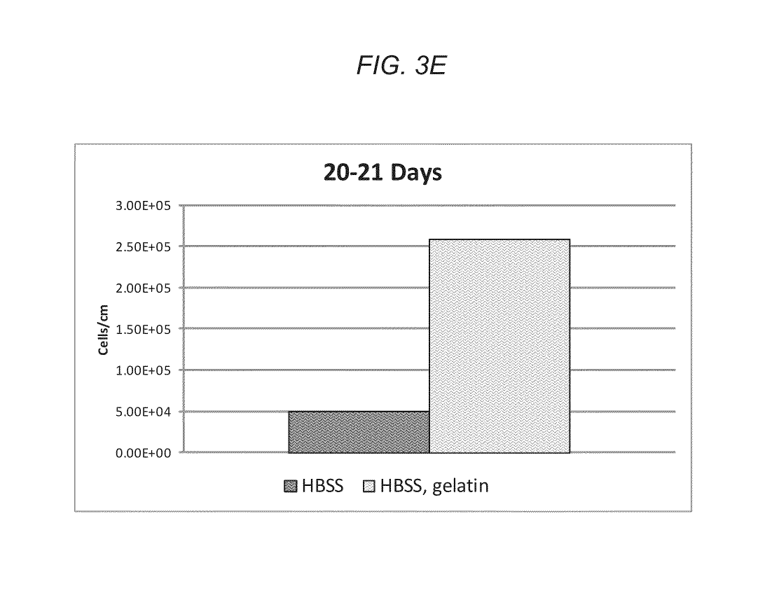

It was found that preservation solutions including hydrolyzed collagen increased the viability of the mesenchymal stem cells harvested from the canine umbilical cords compared to identical solutions without hydrolyzed collagen over time. The results of Example 5 are illustrated in FIGS. 3A-3E. FIG. 3A is a line graph showing the viability of cord samples preserved in different solutions for varying periods of time between one and twenty-one days. As shown in FIG. 3A, samples stored in solutions that included gelatin were better preserved over time than samples stored in identical solutions without gelatin. FIGS. 3B-3E are bar graphs showing the data of FIG. 3A, each respective graph representing different time periods of preservation. Generally, the solutions that did not include gelatin are represented by a lighter bar, and solutions that included gelatin are represented by a darker bar. Similar solutions with and without gelatin are positioned next to each other in the graphs for ease of comparison. FIGS. 3B and 3E show solutions including HBSS with and without gelatin preserved for a period of one day and 20-21 days, respectively. FIGS. 3C and 3D show solutions including HBSS with and without gelatin, as well as solutions including Hibernate, with and without gelatin, that were preserved for a period of six to seven days and 13 days, respectively. As can be appreciated, the samples preserved in solutions including gelatin were observed to have a higher cells/cm count than similar solutions without gelatin stored for a similar period of time. With regard to FIG. 3B, the sample preserved in a solution including HBSS and gelatin had only a slightly higher cells/cm count than the sample preserved in a solution including only HBSS. The slight difference between the two values may be explained by the short one day preservation period during which time cell degradation may not have been as significant of a factor to the viability of the mesenchymal cells.

### Example 6

Mesenchymal cells obtained from canine umbilical cord tissue were used in this example. Three samples of the fresh mesenchymal cells were suspended in Hibernate preservative solution and stored at 4° C. for 6 days. Four samples of the fresh mesenchymal cells were suspended in Hibernate preservative solution with 4 mg/ml hydrolyzed collagen in Hanks balanced salt solution and likewise stored at 4° C. for 6 days. The samples were thawed and two millions cells from each sample were seeded in a 9 cm cell culture dish mesenchymal cell growth media. The samples were harvested after 24 hours and counted to determine a percentage of cells attached by trypsinization and visual counting with trypan blue under regular phase contrast light microscope with counting grid. The results for each sample are shown in Table 6 below:

TABLE 6
Percentage of cells attached in preservation solutions
with and without gelatin

| | | | Average |
|---|---|---|---|
| | | Percentage of cells | percentage of |
| Sample | Solution | attached | cells attached |
| 1 | Hibernate | 26% | 26.33% |
| 2 | | 31% | |
| 3 | | 22% | |
| 4 | Hibernate + | 25% | 41.75% |
| 5 | Hydrolyzed Collagen | 52% | |
| 6 | | 43% | |
| 7 | | 47% | |

It was found that the Hibernate solution that included hydrolyzed collagen increased the percentage of attached cells compared to the identical Hibernate solution without hydrolyzed collagen. For example, the average percentage of cells attached for cells preserved in Hibernate solutions including gelatin was 41.75% compared to 26.33% for cells preserved in Hibernate solutions without gelatin. Mesenchymal cell attachment correlates to cell growth, such that a higher percentage of cell attachment indicates improved growth in the solutions including hibernate and hydrolyzed collagen.

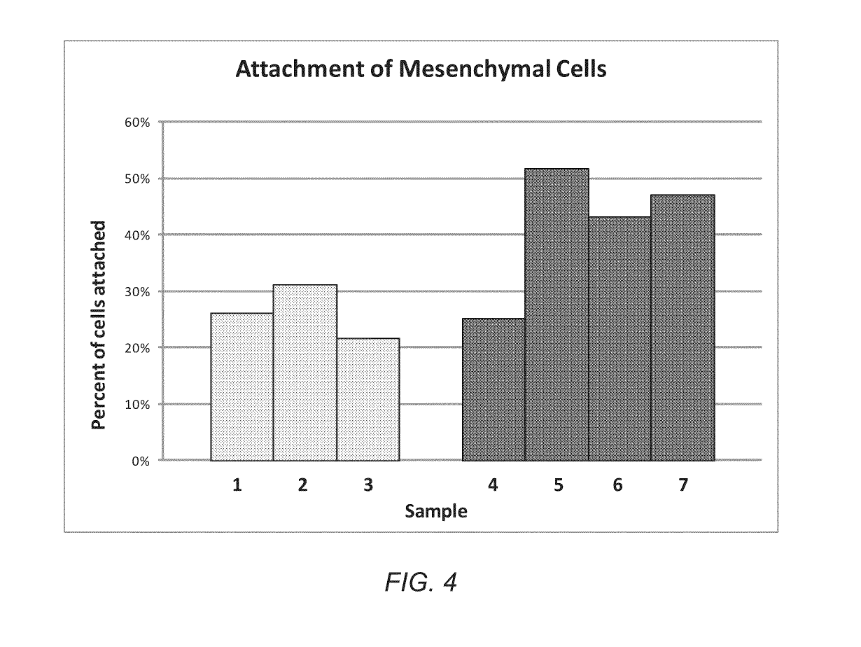

FIG. 4 is a bar graph showing the percentage of cells attached in the samples of Example 6 and Table 6. Each respective bar graph is representative of a sample of Example 6. The samples preserved in Hibernate solutions including hydrolyzed collagen are represented by a dark bar, and samples preserved in Hibernate solutions without gelatin are represented by a light bar.

### Example 7

In this example, chicken embryo hearts were preserved in solutions with and without hydrolyzed collagen. Eleven chicken embryo hearts were harvested from 9-11 day old chicken embryos and then rinsed first with heparin and then a normal saline solution. The hearts were then placed in one of two preservation solutions: a first preservation solution including Wisconsin cold storage solution and a second preservation solution including Wisconsin cold storage solution and 4 mg/ml of hydrolyzed collagen. The hearts were stored in the respective preservation solutions at 4° C. for four days. After this preservation period, each heart was placed onto a chorioallantoic membrane of a live donor chick egg and incubated at 38° C. for 24 hours. The chorioallantoic membranes include vessels which allow for engraftment of the hearts.

The hearts were then evaluated by two blinded assessors and one unblinded assessor for contractility, beats per minute, perfusion regions and perfusion color The assessors evaluated the contractility of the atria and ventricals and rated the contractility on a scale of 0-4 corresponding with contractility states of “none,” “poor,” “fair,” “good,” and “excellent,” respectively. The assessors similarly evaluated the perfusion by determining the number of quadrants of the heart in which perfusion was observed and giving a rating on a scale of 0-4 corresponding to the number of quadrants in which perfusion was observed. Perfusion color was evaluated on a scale of 0-4 corresponding with an assessment of “none,” “poor,” “fair,” “good,” and “excellent,” respectively. The results of the observations are shown in Tables 7-10 below.

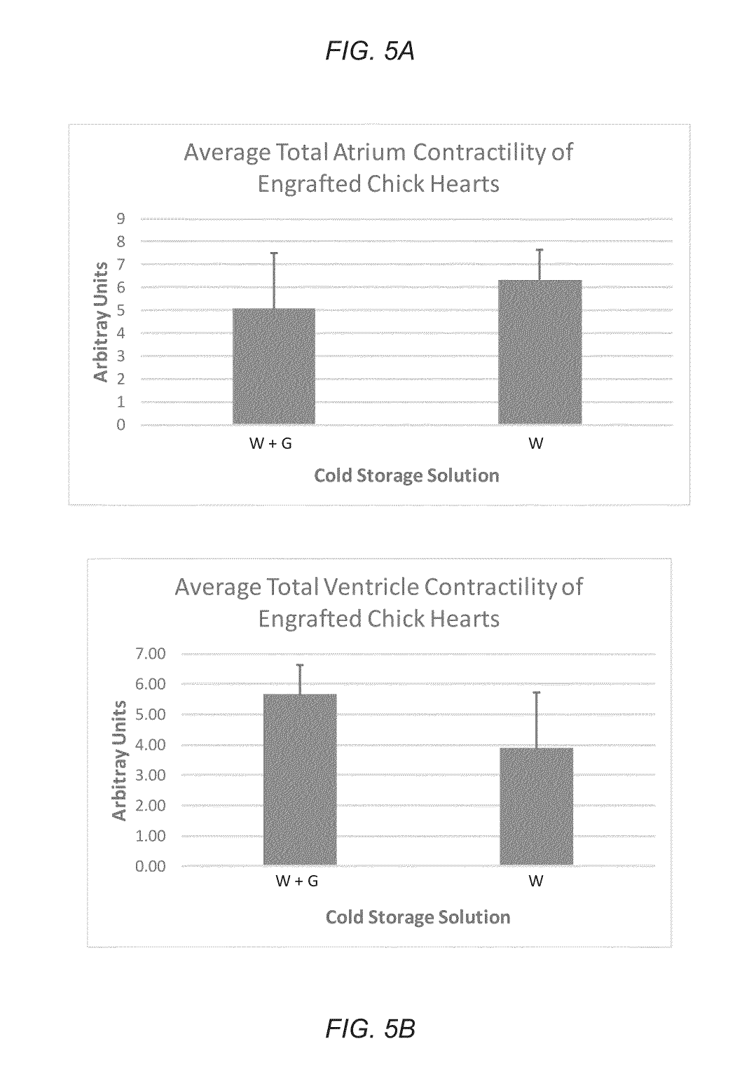

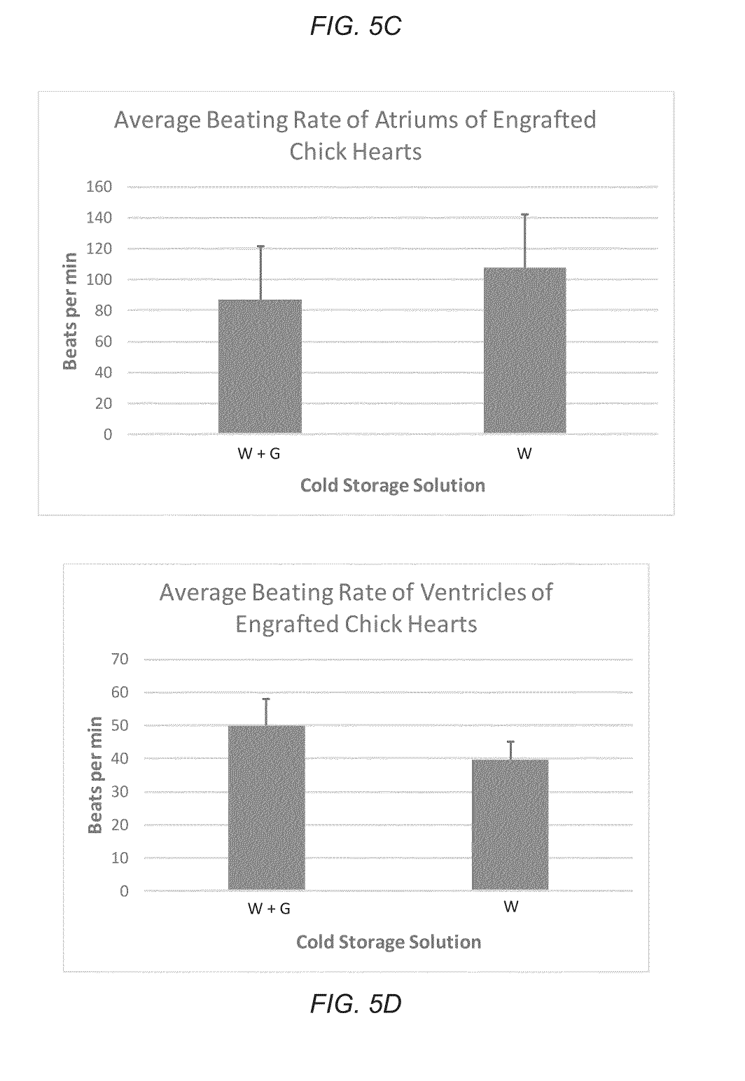

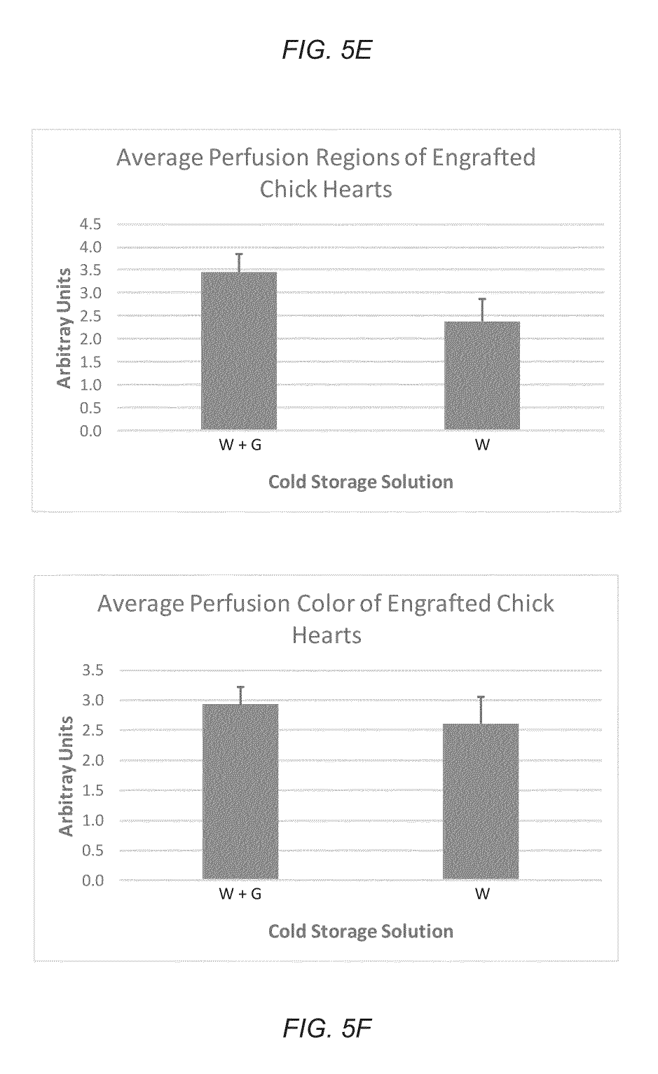

FIG. 5A is a graph showing atrium contractility of the hearts of Example 7 and Table 7. FIG. 5B is a graph showing ventricle contractility of the hearts of Example 7 and Table 8. FIG. 5C is a graph showing atria beats per minute of the hearts of Example 7 and Table 9. FIG. 5D is a graph showing ventricle beats per minute of the hearts of Example 7 and Table 9. FIG. 5E is a graph showing perfusion of the hearts of Example 7 and Table 9. FIG. 5F is a graph showing perfusion color of the hearts of Example 7 and Table 9.

Improved contractility in the ventricals and improved overall perfusion was observed when the hearts were stored in solutions that included gelatin hydrolysate as compared to storage in the same solutions without gelatin hydrolysate. This data is significant because of the important functional role of the ventricles, as compared to the atria which represent a much smaller and less important portion of the heart, and which appeared to have been attempting to compensate for reduced functioning of the ventricles in some cases.

TABLE 7
Atrium contractility of hearts in preservation solutions
with and without gelatin hydrosylate

| | | Right | Left | Total | |
|---|---|---|---|---|---|
| | | atrium | atrium | atrium | Average |
| | | contract- | contract- | contract- | total atrium |
| Sample | Solution | ility | ility | ility | contractility |
| 1 | Wisconsin | 4 | 1 | 5 | 5 |
| 2 | cold storage | 4 | 4 | 8 | |
| 3 | solution + | .33 | 1.33 | 1.66 | |
| 4 | gelatin | 4 | 2.66 | 6.66 | |
| 5 | hydrolysate | 3 | 1 | 4 | |
| 6 | Wisconsin | 3.66 | 3.33 | 6.99 | 6.33 |
| 7 | cold storage | 3.33 | 3.33 | 6.66 | |
| 8 | solution | 2.66 | 3 | 5.66 | |
| 9 | | 3.33 | 3.33 | 6.66 | |
| 10 | | 3.66 | 4 | 7.66 | |
| 11 | | 3 | 1.33 | 4.33 | |

TABLE 8
Ventricle contractility of hearts in preservation solutions
with and without gelatin hydrosylate

| | | Right | Left | Total | Average |
|---|---|---|---|---|---|
| | | ventricle | ventricle | ventricle | total |
| | | contract- | contract- | contract- | ventricle |
| Sample | Solution | ility | ility | ility | contractility |
| 1 | Wisconsin | 3.33 | 2.33 | 5.66 | 5.66 |
| 2 | cold storage | 3 | 3 | 6 | |
| 3 | solution + | 2 | 2.33 | 4.33 | |
| 4 | gelatin | 3.66 | 3.33 | 6.99 | |
| 5 | hydrolysate | 3 | 2.33 | 5.33 | |
| 6 | Wisconsin | 2.66 | 3 | 5.66 | 3.88 |
| 7 | cold storage | 2 | 1.66 | 3.66 | |
| 8 | solution | 2 | 2 | 4 | |
| 9 | | 2.66 | 1.66 | 4.32 | |
| 10 | | 3.33 | 1.33 | 4.66 | |
| 11 | | 0 | 1 | 1 | |

TABLE 9
Atria and ventricle beats per minute of hearts in preservation
solutions with and without gelatin hydrosylate

| | | | Average | | Average |
|---|---|---|---|---|---|
| | | Atria | atria | Ventricles | ventricle |
| Sample | Solution | beats/min | beats/min | beats/min | beats/min |
| 1 | Wisconsin | 113 | 87 | 43 | 50 |
| 2 | cold storage | 111 | | 40 | |
| 3 | solution + | 33 | | 56 | |
| 4 | gelatin | 73 | | 55 | |
| 5 | hydrolysate | 106 | | 56 | |
| 6 | Wisconsin | 98 | 108 | 42 | 40 |
| 7 | cold storage | 167 | | 31 | |
| 8 | solution | 103 | | 40 | |
| 9 | | 115 | | 42 | |
| 10 | | 101 | | 47 | |
| 11 | | 63 | | 36 | |

TABLE 10
Perfusion and perfusion color of hearts in preservation
solutions with and without gelatin hydrosylate

| | | Number of | | | |
|---|---|---|---|---|---|
| | | quadrants | | | Average |
| | | with | Average | Perfusion | perfusion |
| Sample | Solution | perfusion | perfusion | color | color |
| 1 | Wisconsin | 3 | 3 | 3 | 3 |
| 2 | cold storage | 4 | | 2.66 | |
| 3 | solution + | 3.33 | | 3 | 3 |
| 4 | gelatin | 3.66 | | 3.33 | |
| 5 | hydrolysate | 3.33 | | 2.66 | |
| 6 | Wisconsin | 3.33 | 2.39 | 3.33 | 2.61 |
| 7 | cold storage | 2 | | 2.66 | |
| 8 | solution | 2.33 | | 2.66 | |
| 9 | | 2.33 | | 2.33 | |
| 10 | | 2.33 | | 2.66 | |
| 11 | | 2 | | 2 | |

While the invention has been illustrated and described in detail in the drawings and foregoing description, such an illustration and description is to be considered as exemplary and not restrictive in character, it being understood that only various embodiments of the present invention have been shown and described and that all changes and modifications that come within the spirit of the invention are desired to be protected.

What is claimed is:

1. A preservation solution for storage of organs, tissues or cells comprising:
a balanced isotonic solution, and
hydrolyzed collagen.

2. The preservation solution according to claim 1, wherein the balanced isotonic solution comprises sodium, potassium, calcium, magnesium and chloride.

3. The preservation solution according to claim 1, wherein the concentration of hydrolyzed collagen in the solution is between about 1 and about 10 mg/ml.

4. The preservation solution according to claim 1, further comprising at least one antibiotic.

5. The preservation solution according to claim 1, further comprising at least one antimycotic.

6. The preservation solution according to claim 1, further comprising at least one protease inhibitor.

7. The preservation solution according to claim 1, further comprising at least one impermeant anion.

8. The preservation solution according to claim 1, further comprising at least one antifreeze protein.

9. The preservation solution according to claim 1, further comprising at least one synthetic ice blocker.

10. A method for preserving organs, tissue and cells comprising:
harvesting an organ, tissue or cells;
submerging the organ, tissue or cells in a bath of a solution, the solution comprising an aqueous solution of hydrolyzed collagen.

11. The preservation method according to claim 10, wherein the solution further comprises a balanced salt solution.

12. The preservation method according to claim 10, wherein the concentration of hydrolyzed collagen is between about 1 and about 10 mg/ml.

13. The preservation method according to claim 10, further comprising cooling the organ, tissue or cells to 0-37° C.

14. The preservation method according to claim 10, wherein the solution further comprises at least one antibiotic, antimycotic, protease inhibitors, sugar, antioxidants, ion channel drugs, anti-uric acid drug, starch, essential/non-essential amino acids, and/or impermeant ion.

15. The preservation method according to claim 10, wherein the solution further comprises at least one antifreeze protein, and/or ice blocking agent.

16. A kit configured to preserve organs, tissue or cells comprising:
A container configured for storing an organ, tissue, or cells;
a preservative solution comprising an aqueous solution of hydrolyzed collagen.

17. The kit of claim 16 wherein the preservative solution further comprises a balanced salt solution.

18. The kit according to claim 16, further comprising an insulated container configured to store organs, tissue or cells.

19. The kit according to claim 16, wherein the concentration of hydrolyzed collagen is between about 1 and about 10 mg/ml.

20. The kit according to claim 16, wherein the solution further comprises at least one antibiotic, antimycotic, protease inhibitor, and/or impermeant ion.

21. The kit according to claim 16, wherein the solution further comprises at least one antifreeze protein, and/or ice blocking agent.

22. A method for transplantation of an organ, tissue or cells comprising:
removing the organ, tissue, or cells from a preservative solution, the preservative solution comprising an aqueous solution of hydrolyzed collagen;
injecting or implanting the organ, tissue, or cells into a recipient.
